(19) United States
(12) Patent Application Publication
(10) Pub. No.: US 2026/0007623 A1
(43) Pub. Date: Jan. 8, 2026

(54) METHOD TO INDUCE SENESCENCE IN GLIOBLASTOMA MULTIFORME

(71) Applicant: University of South Florida, Tampa, FL (US)

(72) Inventors: Mildred Enid Acevedo-Duncan, Tampa, FL (US); Grazielly Caroline Teodoro, Tampa, FL (US)

(21) Appl. No.: 19/260,020

(22) Filed: Jul. 3, 2025

Related U.S. Application Data
(60) Provisional application No. 63/667,462, filed on Jul. 3, 2024.

(51) Int. Cl. A61K 31/185; A61P 35/00

(52) U.S. Cl. CPC A61K 31/185; A61P 35/00

(57) ABSTRACT

Disclosed are methods, compounds, and compositions for treating glioma and glioblastoma using a Protein kinase C ζ inhibitor, ζ-Stat. Provided are new therapies using targeted ζ-Stat inhibitors that specifically disrupt key pathways involved in glioma and glioblastoma progression.

### CROSS REFERENCE TO RELATED APPLICATION

This application claims the benefit of U.S. Provisional Application Ser. No. 63/667,462, filed Jul. 3, 2024. The entire content of which is incorporated by reference in its entirety.

### BACKGROUND

Glioblastoma multiforme (GBM) is the most aggressive form of brain cancer, classified as grade IV. It originates from astrocytes, star-shaped glial cells that provide support to nerve cells [1]. The incidence of GBM is approximately 3.19 cases per 100,000 person-years [2]. Despite advances in therapy, the prognosis for GBM remains poor, with a median overall survival of 15 months following diagnosis [3].

Treatment typically involves a combination of surgery, radiation therapy, and chemotherapy with temozolomide. However, the effectiveness of these treatments is hindered by several factors, including the blood-brain barrier, tumor infiltration, and the genomic diversity of GBM cells [4]. This complexity makes developing and implementing effective targeted therapies a challenge, highlighting the critical need for innovative therapeutic approaches.

Protein kinase C (PKC) is a family of at least 12 serine/threonine kinases that play a critical role in cellular signaling. PKCs are involved in several essential cellular processes, including cell proliferation, differentiation, apoptosis, and angiogenesis [5]. Based on their structural and activation characteristics, PKCs are classified into three major groups: conventional/classical PKC isozymes (cPKCs; α, βI, βII, and γ), novel PKC isozymes (nPKCs; δ, ε, η, and θ), and atypical PKC isozymes (aPKCs; ζ, ι, and λ). Atypical PKCs (aPKCs), activated by dysregulated upstream phosphatidylinositol 3-kinase (PI3K) signaling, may serve as promising therapeutic targets for adjuvant therapy in the treatment of glioblastoma [6].

Among the different isoforms of protein kinase C, PKC-ζ (PKC-zeta) plays a significant role in driving glioblastoma proliferation, migration, and invasion by regulating cytoskeletal rearrangement and cell adhesion. Due to its involvement in these critical cellular processes, reducing PKC-ζ expression may impair the migratory ability of glioblastoma cells, presenting it as potential target for therapeutic approaches aimed at limiting GBM progression [7-8].

Connexins (Cxs) are integral membrane proteins that assemble into hexameric structures known as connexons, or hemichannels, which are embedded in the plasma membrane. When aligned with connexons from adjacent cells, these structures form gap junctions' intercellular communication (GJIC) channels that allow for the direct exchange of ions, metabolites, and small signaling molecules. GJIC is essential for regulation of normal cell function and development, tissue homeostasis, growth, and differentiation. Nevertheless, abnormal function of GJIC and reduction of cell-coupling via GJS is associated with cancer development [9, 20].

Connexin 43 (Cx43) is among the most widely expressed connexins in the central nervous system and plays a critical role in mediating communication between glioma cells and surrounding astrocytes. Besides their canonical role facilitating cell-cell communication, Cx43 contributes to increased cell adhesion and has been implicated in facilitating tumor cell migration [10, 11, 15]. Additionally, Cx43 regulates tumor cell plasticity, since the overexpression of Cx43 in glioma cells can induce a phenotypic shift from a mesenchymal to an epithelial morphology [10, 15]. Furthermore, elevated levels of Cx43 in glioma cells have been associated with increased resistance to the chemotherapeutic agent temozolomide (TMZ), while Cx43 knockdown has been shown to sensitize these cells to treatment. This effect is thought to be mediated, at least in part, through modulation of the PI3K signaling pathway, modulated by Cx43 expression activation [13, 14].

Phosphorylation of Cx43 at serine residues (Ser365, Ser368, Ser369, Ser372, Ser373) by aPKCs has been linked to tumor progression. Phosphorylation at Ser368 plays a critical role in cell cycle regulation, particularly during the S and G2/M phases of mitosis. In addition, this post-translational modification disrupts the assembly of gap junctions and promotes Cx43 internalization, thereby impairing its essential role in intercellular communication [16-19]. The elevated levels of phosphorylated Cx43 have been associated with enhanced tumor invasiveness in various cancers, including breast and pancreatic cancer, and are associated with poor prognosis in gliomas [21-23]. As a result, inhibiting Cx43 phosphorylation is being investigated as a potential strategy to restore gap junction function and counteract tumor progression.

The PI3K/AKT (Phosphoinositide 3-Kinase/Protein Kinase B) pathway plays a central role in the pathogenesis and treatment resistance of glioblastoma. Activation of this pathway promotes tumor cell growth, survival, and invasion, often through mutations or deletions in upstream regulators such as PTEN (Phosphatase and Tensin Homolog), a tumor suppressor commonly mutated in GBM. Cx43 contributes to this oncogenic signaling by activating PI3K/AKT pathway. Notably, this pathway also regulates atypical PKC isoforms, including PKC-ζ, which is a key modulator of cytoskeletal organization, migration, and gap junction communication disruption by Cx43 phosphorylation. The reciprocal amplification between PI3K activation and Cx43 phosphorylation establishes a feedback mechanism that further entrenches resistance to therapy. Consequently, targeting elements of the PI3K axis, including downstream proteins such as PKC-ζ holds significant promise as a therapeutic strategy for overcoming treatment resistance in GBM [14, 24, 25, 26].

The downregulation of PI3K/AKT signaling leads to reduced activation of Cx43, which has been reported to be implicated in the transcriptional regulation of N-cadherin, a protein involved in epithelial-mesenchymal transition (EMT). Consequently, N-cadherin and phospho-vimentin expression levels are significantly reduced, disrupting the morphological shift from epithelial to mesenchymal phenotype. As a result, there is a reduction in migratory potential of glioblastoma multiforme cells [27-29]. There is need to develop new therapeutic strategies that can effectively address some of the challenges in GBM treatment.

### SUMMARY

A first aspect of the disclosure is a method of treating glioma glioma or glioblastoma comprising administering to a subject in need an effective amount of a phosphokinase C ζ (PKC-ζ) inhibitor, wherein the PKC-ζ inhibitor is 8-hydroxy-1,3,6-naphthalenetrisulfonic acid (ζ Stat).

A second aspect is a method of reducing or inhibiting the proliferation of glioma or glioblastoma cells, comprising delivering to a glioma or glioblastoma cell an effective amount of the PKC-ζ inhibitor ζ Stat.

A third aspect is a method of inhibiting the expression of PKC-ζ in a glioma or glioblastoma cell comprising delivering to the cell an effective amount of a PKC-ζ inhibitor inhibitor ζ Stat.

### BRIEF DESCRIPTION OF THE DRAWINGS

FIG. 1 illustrates the chemical structure of ζ-Stat (8-hydroxy-1,3,6-naphthalenetrisulfonic acid).

FIG. 2 represents the effects of ζ-Stat in U-87MG cell proliferation.

FIG. 3 represents the effects of ζ-Stat in T98G cell proliferation.

FIG. 4 illustrates expression levels of p-PKC-ζ, PKC-ζ, p-Cx43 and Cx43 in T98G and U-87MG treated samples.

FIG. 5 shows co-immunoprecipitation of PKC-ζ using Cx43 as a probe. Western blotting analysis illustrates the association between the proteins.

FIG. 6 shows the flow cytometry analysis to assess apoptosis in T98G and U-87MG cell lines treated with ζ-Stat.

FIGS. 7A-7B show evaluation of senescence in T98G and U-87MG cell lines. FIG. 7A shows T98G and U-87MG cell lines treated with ζ-Stat stained with β-Galactosidase (blue) for the evaluation of senescence at 10× magnification. FIG. 7B shows expression levels of the senescence markers, Lamin B1 and β-Galactosidase, in T98G and U-87MG cell lines treated with ζ-Stat.

FIG. 8 shows expression levels of proteins involved in cell progression in T98G and U-87MG cell lines treated with ζ-Stat.

FIG. 9 shows expression levels of PI3K/AKT signaling in T98G and U-87MG cell lines treated with ζ-Stat.

FIG. 10 shows expression levels of EMT related proteins in T98G and U-87MG cell lines treated with ζ-Stat.

### DETAILED DESCRIPTION

This disclosure introduces new approaches for glioma and/or glioblastoma multiforme (GBM) treatment using ζ-Stat, an atypical protein kinase C (aPKC) inhibitor that targets the PKC-ζ isoform. As a PKC-ζ inhibitor (aPKC inhibitor), ζ-Stat has demonstrated promising results in reducing the expression of their respective target, which play crucial roles in tumor growth and survival. Specifically provided are methods, compounds, and compositions for treating glioma and/or GBM through the administration of effective amounts of a PKC-ζ inhibitor ζ-Stat.

The disclosed aspects offer an alternative for treating glioma, glioma tumor, glioblastoma multiforme or glioblastoma, one of the most aggressive and difficult brain cancers to treat. Given the limited effectiveness of current treatments and the poor prognosis often associated with glioblastoma cancer, there is a critical need for new therapies that can better target the underlying mechanisms driving tumor growth and resistance. This disclosure fills that gap by providing targeted inhibitors that specifically disrupt key pathways involved in glioma and glioblastoma progression, potentially improving patient outcomes.

### Methods

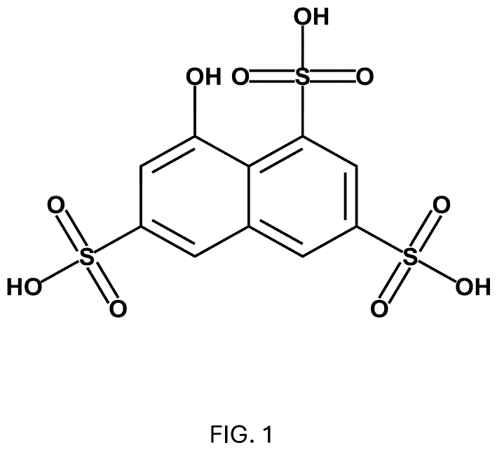

One aspect is the novel use of ζ-Stat (8-hydroxy-1,3,6-naphthalenetrisulfonic acid), shown in FIG. 1, as treatment for glioma, and glioblastoma or glioblastoma cancer. The disclosed compounds have demonstrated anti-proliferative effects against glioma and glioblastoma cells. In addition, the disclosed compounds also demonstrated the ability to reduce the phosphorylation of Connexin 43 by PKC-ζ through downregulation of PI3K/AKT, thereby inducing senescence and inhibiting EMT. An aspect is a method of treating glioma and/or glioblastoma comprising administering to a subject in need an effective amount of a protein kinase C-ζ (PKC-ζ or PKC-zeta) inhibitor, wherein the PKC-ζ inhibitor is 8-hydroxy-1,3,6-naphthalenetrisulfonic acid (ζ Stat). The effective amount of a protein kinase C-ζ inhibitor may be a therapeutically effective amount.

The expressions “treatment” and “treating” are used synonymously herein and refer broadly to any action taken to produce a positive effect. This may involve, but is not limited to, completely resolving, or improving the symptoms of a disease, delaying the onset or progression of a disease, preventing its occurrence in individuals at risk, or improving the quality of life of an individual affected by a condition.

The term “cancer” as used herein encompasses abnormal cellular proliferation, including both benign and malignant growths. Such conditions are characterized by uncontrolled division of cells, potential spread to other tissues, and traits such as evasion of apoptosis, instability within the genome, and disruptions in cellular signaling networks.

A “therapeutically effective amount” refers to a quantity of a compound sufficient to achieve the intended therapeutic benefit, such as suppressing tumor growth or influencing the function of a target protein. The appropriate dosage may differ based on factors including the Subject's weight, age, the seriousness of the disease, the formulation used, and the route by which the compound is administered.

The term “subject” as used herein refers to any animal organism, including both human and non-human species. This includes mammals commonly involved in research studies or veterinary contexts. In certain embodiments, the subject is a human individual requiring therapeutic intervention.

The “subject in need” or “subject in need of treatment” may be an individual diagnosed with glioblastoma multiforme, a malignant primary brain tumor characterized by aggressive growth and poor prognosis. Diagnosis of glioma or glioblastoma may be made using standard clinical techniques including magnetic resonance imaging (MRI), computed tomography (CT), and histopathological confirmation via biopsy.

In the context of this disclosure, the term “inhibitor” refers to chemical compound, molecular entity, or biologically derived agent that disrupts or reduces the normal function of a specific target protein. This interference may involve direct suppression of the protein's enzymatic activity or regulation of its expression. The specific mechanism of inhibition is dependent on the functional role and biological context of the target protein.

As used herein, the term “PKC inhibitor” refers to any agent that reduces or blocks the activity or expression of one or more protein kinase C (PKC) isoforms, including classical, novel, and atypical forms. This definition encompasses compounds that selectively target atypical isoforms, such as PKC-ζ. Examples of such inhibitors include, but are not limited to, ζ-Stat, ICA-1, ACPD, fludarabine, auranofin, thimerosal, phenylmercuric acetate, ebselen, cisplatin, taxol, apomorphine, ellagic acid, and pyrantel pamoate. These inhibitors may consist of small molecules, peptides, or biologically derived substances that influence PKC function either through direct interaction or via indirect regulatory pathways.

The term “PKC-ζ inhibitor” refers to agents that inhibit the catalytic activity or expression of PKC-ζ. One example is 8-hydroxy-1,3,6-naphthalenetrisulfonic acid (ζ-Stat), which has been shown to modulate the activity of PKC-ζ selectively. Similar to PKC-ι inhibitors, PKC-ζ inhibitors may function through multiple mechanisms, including ATP-competitive inhibition, conformational changes, or interference with substrate recognition.

The present disclosure relates to novel therapeutic applications of ζ-Stat for the management of glioma or glioblastoma multiforme. In one embodiment, the method comprises administering a therapeutically effective dose of ζ-Stat to a subject requiring such treatment, for example over a period of time, such as a period of five consecutive days. In some aspects, the ζ-Stat is provided in a composition. In some aspects, the composition is a pharmaceutical composition that includes one or more pharmaceutically acceptable carrier or adjuvant. An aspect is a pharmaceutical composition that includes a therapeutically effective amount or quantity of ζ-Stat formulated with one or more pharmaceutically acceptable carrier or adjuvant.

The phrases “pharmaceutically acceptable carrier, “pharmaceutically acceptable excipient,” or “pharmaceutically acceptable adjuvant” refer to any substance that facilitates the effective delivery of a therapeutic agent and is compatible with the active ingredient. Examples include, but are not limited to, solvents, dispersing media, coating substances, antimicrobial preservatives, isotonicity adjusters, and agents that modulate absorption rates. The utilization of such carriers in drug formulations is well recognized within the pharmaceutical field. Any carrier or excipient commonly used by those skilled in the art may be incorporated, so long as it does not interfere negatively with the stability or function of the active compound(s), including the PKC inhibitor described herein.

Another aspect is a method of reducing or inhibiting the proliferation of glioma or glioblastoma cells, comprising administering to a glioma or glioblastoma cell an effective amount of a PKC-ζ inhibitor. In the disclosed aspects, the PKC-ζ inhibitor is 8-hydroxy-1,3,6-naphthalenetrisulfonic acid (ζ-Stat). The ζ-Stat PKC-ζ inhibitor may have the formula as depicted in FIG. 1. The disclosure demonstrates the anti-proliferative effect of ζ-Stat against glioma and glioblastoma multiforme cells. For example, ζ-Stat at 40 μM decreased proliferation by 30% (p≤0.001) in U-87MG cells. In T98G cells, ζ-Stat at 80 μM decreased T98G proliferation in about 40% (p≤0.001). These findings show that ζ-Stat can effectively reduce cell proliferation in glioblastoma cell lines. Furthermore, concentrations of 40 μM and 80 μM ζ-Stat were selected as the working doses for experiments using U-87MG and T98G cell lines, respectively.

Various analytical methods are available and routinely utilized by those skilled in the art to determine the expression levels of PKC-ζ. Representative techniques include, but are not limited to, Western blotting, enzyme-linked immunosorbent assay (ELISA), and radioimmunoassay (RIA). The procedures for performing such assays are well known and widely accessible in the field. Additional techniques suitable for detecting or quantifying protein levels may likewise be employed, as would be readily appreciated by a person of ordinary skill in the art, and are intended to fall within the scope of the present invention.

In accordance with the methods described herein, methods of assessing the likelihood of a subject responding to a therapeutic regimen targeting PKC-ζ in glioblastoma treatment are provided, and a control sample is incorporated for comparative purposes. A “control” sample or value refers to a sample that serves as a reference, usually a known reference, for comparison to a test sample. Such a control sample may include, without limitation, glioma cells derived from an individual of the same species as the subject who does not display glioblastoma pathology; glioma cells obtained from the subject prior to glioblastoma development; or glioma cells from a subject identified as non-responsive to therapies based on PKC-ζ inhibition. An aspect is a method of assessing the likelihood of a subject responding to a therapeutic regimen targeting PKC-ζ in glioblastoma comprising delivering or administering a PKC-ζ inhibitor, for example (Stat, to a glioma or glioblastoma cell. The glioma or glioblastoma cell ma be from the subject or a control sample of glioma cells.

The phrases “a subject identified as non-responsive” or “a subject characterized as non-responsive” refer to an individual who, following administration of glioblastoma therapy, fails to demonstrate the intended therapeutic outcomes. Such outcomes include, without limitation, reduction, delay, or prevention of glioblastoma symptoms, improvement in quality of life, and/or extension of overall survival.

A further aspect is a method of reducing expression of PKC-ζ and Connexin 43 (Cx43) in glioblastoma cell by exposing or delivering ζ-Stat to the cells. Connexin 43, a gap junction protein, is known for its significant involvement in glioblastoma progression, invasion, and resistance. In embodiments, Cx43 phosphorylation is inhibited by ζ-Stat, leading to restored gap junction function and reduced tumor progression in glioblastoma. The inventors show that treatment of T98 cells with ζ-Stat at a concentration of 80 M resulted in a reduction of phosphorylated PKC-ζ (p-PKC-ζ) levels by approximately 28%, total PKC-ζ by 12%, phosphorylated Connexin 43 (p-Cx43) by 27%, and total Connexin 43 (Cx43) by 52%. In U-87MG cells, ζ-Stat treatment led to decreases of p-PKC-ζ by 6%, total PKC-ζ by 18%, p-Cx43 by 13%, and total Cx43 by 10%.

In disclosed aspects, ζ-Stat induces cellular senescence in glioma and glioblastoma cells. The inventors show in the Examples that ζ-Stat induced cellular senescence in T98G and U-87MG cells, evidenced by the increase of β-galactosidase staining in the treated cells compared to control, as well as increased expression of senescence-associated proteins, such as β-galactosidase. In addition, ζ-Stat disrupts nuclear membrane integrity associated with senescence of cells. Delivery of is shown to downregulate nuclear membrane proteins, for example Lamin B1, in glioblastoma cells. These findings highlight the cell line-specific mechanisms of action of the inhibitors, while supporting the broader concept of disrupting GBM growth through PKC-ζ inhibition. An aspect is a method of inducing senescence in glioma or glioblastoma cells and GBM cancer using the compound ζ-Stat. The method comprises administering to a subject in need thereof a therapeutically effective dose of ζ-Stat to treat the glioma or GBM. In certain aspects, the therapeutically effective dose of ζ-Stat ranges from approximately 40 M to 80 M, or 40 μM, 50 M, 60 μM, 70 μM or 80 μM.

Another aspect includes downregulating the PI3K/AKT signaling pathway in glioma or glioblastoma cell by delivering ζ-Stat to the cells. The PI3K/AKT signaling pathway is implicated in chemotherapy resistance. Western blot analysis has been used to demonstrate that ζ-Stat downregulates the PI3K/AKT signaling pathway. For example, in T98G glioblastoma cells, treatment with ζ-Stat at 80 M resulted in a 27% reduction in PI3K expression and a modest 6% decrease in AKT levels. Also in U-87MG cells, ζ-Stat at 40 μM decreased PI3K expression by 17% and AKT levels by 33%. A disclosed aspect is a method of inhibiting the PI3K/AKT signaling pathway by delivering ζ-Stat to glioblastoma cells.

In the disclosed aspects, inhibition of the PI3K/AKT pathway by ζ-Stat leads to decreased activation of Connexin 43, which in turn causes a significant disruption in Epithelial-Mesenchymal Transition processes, as shown by reduction in the expression of N-cadherin and phosphorylated vimentin. This downregulation impacts the migratory and epithelial to mesenchymal transition of glioblastoma cells. Accordingly, the disclosure demonstrates direct effects on GBM signaling pathways involved in tumor proliferation, progression, and migration. Targeting PKC-ζ provides a strategy to suppress multiple signaling pathways concurrently, thereby enhancing therapeutic effectiveness. In embodiments, ζ Stat reduces the migratory and invasive characteristics of glioblastoma cells, for example limiting or inhibiting migration of GBM cells or epithelial to mesenchymal transition of glioblastoma cells.

### Compositions

Another aspect relates to a composition, therapeutic composition, or pharmaceutical composition containing an effective amount or dose of a PKC-ζ inhibitor compound, e.g., ζ Stat. A pharmaceutical composition may be a PKC-ζ inhibitor compound, e.g., ζ Stat, combined with a pharmaceutically acceptable carrier. The composition can be prepared in various forms such as oral tablets or capsules, injectable solutions, or extended-release formulations, tailored according to the desired method of administration. Possible delivery routes include oral intake, intravenous injection, or direct administration to the brain, chosen based on the subject's needs. The formulation may also comprise additives like stabilizers, solubilizing agents, or preservatives to improve the drug's stability, effectiveness, or shelf life. This composition is intended to inhibit PKC-ζ activity in glioma or glioblastoma cells, thereby slowing tumor progression or improving the outcomes of other treatment modalities.

The compositions may be delivered or administered alone or in combination with existing therapies, such as temozolomide, radiotherapy, or surgical resection. Administration routes may include intravenous, oral, intratumoral, or intracranial delivery, depending on the formulation and clinical requirements. Furthermore, patients may be stratified based on expression levels of PKC-ζ or phosphorylated Cx43 in tumor biopsy samples, allowing for personalized treatment regimens that enhance response and reduce unnecessary toxicity.

Disclosed is a novel and effective approach to treating glioblastoma multiforme through targeted inhibition of PKC-ζ. By reducing PKC-ζ activity, blocking the phosphorylation of Connexin 43, modulating senescence, downregulating PI3K/AKT signaling pathway, and reducing EMT, this strategy addresses the core mechanisms that drive GBM malignancy. The selective inhibitors described herein may serve as standalone or adjunct therapies in the management of GBM, offering a promising avenue to overcome the limitations of current treatment options.

### Miscellaneous

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter.

In this application, singular terms such as “a,” “an,” and “the” are intended to include the plural forms. Accordingly, references to “a compound” or “a treatment” should be understood to include one or more compounds or treatments.

The terms “includes,” “including,” “has,” “having,” “with,” and comparable expressions are to be construed in an open-ended manner, permitting the presence of additional elements, components, or steps beyond those explicitly recited. Such terms are to be interpreted in a manner consistent with the term “comprising.” Embodiments recited as “including,” “comprising,” or “having” certain elements are also contemplated as “consisting essentially of” and “consisting of” those certain elements.

The terms “about” or “approximately,” as applied to measurable values, are intended to cover variations that would be deemed reasonable by those skilled in the art. Such variations may arise from typical experimental inaccuracies, measurement constraints, or inherent biological differences, and can include deviations in the range of ±1-20%, or other ranges commonly accepted within the relevant field. In the context of biological systems, “about” may also encompass differences up to an order of magnitude, often within a two- to five-fold range, and preferably within a two-fold range. Unless otherwise specified, the term “about” should be understood to represent a value falling within an acceptable margin of error.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

Glioblastoma multiforme (GBM) is the most aggressive form of brain cancer, classified as grade IV. It originates from astrocytes, star-shaped glial cells that provide support to nerve cells. GBM presents within a complex network of blood vessels, showing notable heterogeneity at the transcriptional and genomic levels. This diversity poses a challenge for targeted therapy approaches. Despite current treatment modalities such as surgical resection, radiation therapy, and chemotherapy with temozolomide, GBM patients typically face a survival window of 8 to 12 months post-diagnosis. The inventors aimed to enhance understanding of GBM pathogenesis by investigating the effects of novel therapeutic strategies on glioblastoma cells. In this disclosure the effects of atypical protein kinase C (aPKC) inhibitor, ζ-Stat (8-hydroxy-1,3,6-naphthalenetrisulfonic acid), a specific inhibitor of Protein Kinase C-ζ (PKC-ζ) in GBM cell lines (see FIG. 1).

The effects of ζ-Stat on GBM was evaluated targeting Phospho-PKC-ζ (p-PKC-ζ), PKC-ζ, Phospho-Connexin 43 (p-Cx43), and Cx43. For example, ζ-Stat was assessed U-87MG and T98G glioblastoma cell lines, with particular emphasis on the Phosphoinositide 3-Kinase/Protein Kinase B (PI3K/AKT) pathway, which influence Cx43 phosphorylation levels, impacting Epithelial-Mesenchymal Transition (EMT) marker expression. This novel therapeutic strategy is shown to address some of the challenges in GBM treatment.

Multiple experimental approaches were employed to assess outcomes, including proliferation assays, western blot analysis, flow cytometry, co-immunoprecipitation, and 0-galactosidase staining. ζ-Stat demonstrated the ability to inhibit GBM proliferation significantly, inducing senescence of T98G and U-87MG cells. The inhibitors decreased the expression of the levels of PKC-ζ and p-Cx43. Furthermore, ζ-Stat demonstrated the ability to downregulate the PI3K/AKT pathway, directly affecting GBM progression and EMT. These findings demonstrate that ζ-Stat may be potential treatment for GBM cancer.

### Materials and Methods

ζ-Stat was obtained from National Institute of Health—NIH (Bethesda, JVID. The drugs were dissolved in sterile distilled water (vehicle) before use. Primary antibodies were obtained as follows: PKC-ζ (sc-17781, Santa Cruz Biotechnology, Dallas, TX, USA), beta actin peroxidase (A3854, Millipore-Sigma, Burlington, MA, USA), phospho-PKC-ζ (9378S, cell signaling, Danvers, MA, USA), Connexin 43 (3512S, cell signaling, Danvers, MA, USA), phospho-connexin 43(3511S, cell signaling, MA, USA), β-Gal Antibody (sc-377257, Santa Cruz Biotechnology, Dallas, TX, USA), Lamin B1 (sc-374015, Santa Cruz Biotechnology, Dallas, TX, USA), p27 (2552S, cell signaling, MA, USA), p21 (2947S, cell signaling, MA, USA), Cyclin D1 (55506S, cell signaling, MA, USA), p21 (2947S, cell signaling, MA, USA), CDK4 (12790S, cell signaling, MA, USA), PI3 Kinase p55 (11889S, cell signaling, MA, USA), AKT (2920S, cell signaling, MA, USA), N-Cadherin (4061S, cell signaling, MA, USA), Phospho-vimentin (3877S, cell signaling, MA, USA). Anti-rabbit or anti-mouse antibodies conjugated with horseradish peroxidase (HRP) were used as secondary antibodies.

Enhanced chemiluminescence (ECL) reagents, such as the SuperSignal™ West Pico Chemiluminescent Substrate (Product No. 34080), were obtained from Pierce Biotechnology (Rockford, IL, USA). Dulbecco's phosphate-buffered saline (DPBS) lacking magnesium and calcium ions (Product No. D8537), and Trypsin-EDTA (Ethylenediaminetetraacetic acid) solution (Product No. T4049), were obtained from Sigma-Aldrich (St. Louis, MO, USA). Senescence β-Galactosidase staining kit was obtained from Cell Signaling (Trask Lane Danvers, MA, USA). PKC-ζ conjugated to agarose was obtained from Santa Cruz Biotechnology (sc-17781, Dallas, TX, USA).

### Cell Culture, Media, Cell Lines and Culture Conditions

T98G (ATCC CRL-1690™) and U-87MG (ATCC HTB-14™) GBM cell lines were cultured at 37° C. in a 5% CO₂. Both cell lines were maintained in Eagle's Minimum Essential Medium (EMEM; ATCC 30-2003™), supplemented with 10% fetal bovine serum (FBS, v/v) and 5 μg/mL penicillin. Cells were seeded into T75 culture flasks and grown until approximately 80% confluency. Upon reaching confluency, cells were detached using Trypsin-EDTA, neutralized with an equal volume of complete EMEM, and subsequently used for experiments.

### Statistical Analysis

Statistical analysis was performed using GraphPad Prism 10.3.1. version. Treated samples were compared to control using one-way ANOVA with Dunnett's multiple comparisons. Mean±SEM (standard error of means) of three independent trials were represented included in the bar graphs presented in this invention.

### Example 1: Cell Proliferation Assay

To assess the proliferation of T98G or U87-MG cell lines, cells were treated with varying doses of ζ-Stat for 5 days in T75 flasks, with approximately 100,000 cells seeded per flask. Control flasks, without any drug treatment, were also cultured for each condition. After the treatment period, the cells were detached using Trypsin-EDTA (3 mL/flask), neutralized with an equal volume of EMEM medium, and counted using a hemocytometer.

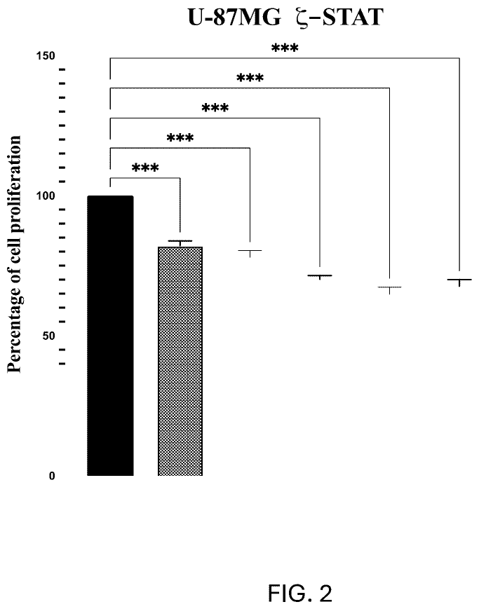

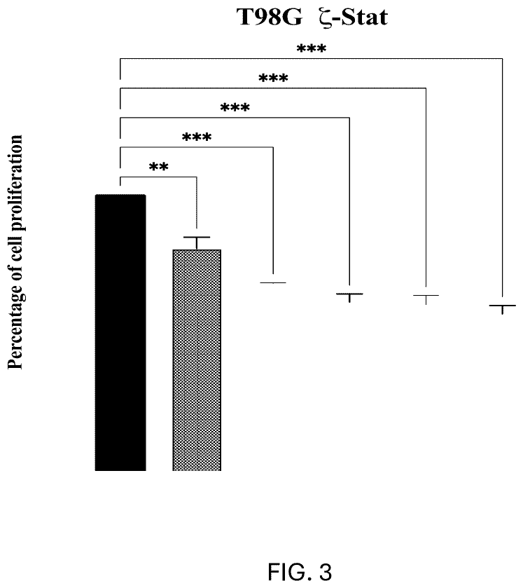

ζ-Stat demonstrated anti-proliferative effects in T98G and U-87MG cell lines. ζ-Stat (40 μM) decreased cell proliferation by 30% (p≤0.001) in U-87MG cells (FIG. 2). In T98G cells, ζ-Stat (80 μM) decreased T98G proliferation about 40% (p≤0.001) (FIG. 3). These findings suggest that ζ-Stat can effectively reduce cell proliferation in glioblastoma cell lines. In addition, ζ-Stat (40 μM) was determined to be the working concentration for further experiments with U-87MG cells, while ζ-Stat (80 μM) was selected for experiments with T98G cells.

### Example 2: Preparation of Cell Lysates and Western Blotting

Cells were seeded in 100 mm cell culture plates and were grown to 40-50% confluence prior to the treatment. Five days post-treatment with ζ-Stat, cell lysates were collected with lysis buffer (C7027, Invitrogen, Waltham, MA, USA), sonicated and centrifuged. The protein concentration in each sample was determined using the Bradford Assay, performed using albumin standard and dye (Bio-Rad protein assay dye reagent concentrate). For Western blot analysis, 100 μg of protein from each cell lysate was loaded onto polyacrylamide gels and separated by SDS-PAGE (Sodium Dodecyl Sulfate-Polyacrylamide Gel Electrophoresis). Subsequently, the proteins were transblotted onto a 0.2 μM nitrocellulose membrane, followed by incubation with a 4% milk blocking solution. Primary antibody was added and incubated overnight, followed by the addition of secondary antibody. The intensity of the bands was analyzed using the Amersham Imager 600 software (GE Healthcare, Chalfont St Giles, UK). Protein expression levels were normalized with loading control of beta-actin peroxidase.

### Example 3: Effect of ζ-Stat on PKC-ζ and Cx43 Levels in Glioblastoma Multiforme

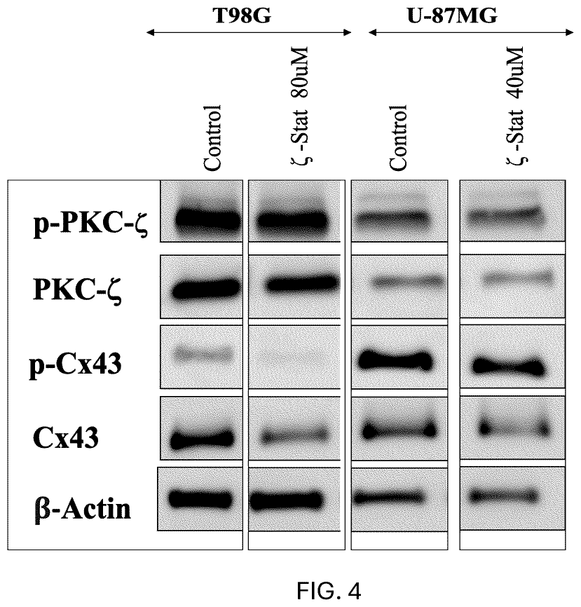

Western blots were performed to investigate the effects of ζ-Stat on the expression of PKC-ζ and Cx43 in glioblastoma cell lines. As illustrated in FIG. 4, ζ-Stat treatment of T98G cells resulted in a reduction of p-PKC-ζ by 28%, total PKC-ζ by 12%, p-Cx43 by 27%, and total Cx43 by 52%. In U-87MG cells, ζ-Stat induced a decrease of p-PKC-ζ by 6%, total PKC-ζ by 18%, p-Cx43 by 13%, and total Cx43 by 10%. β-actin served as a loading control in these experiments.

### Example 4: Co-Immunoprecipitation Confirmation of PKC-ζ and Cx43 Association

Cx43 was co-immunoprecipitated in protein samples of U-87MG or T98G with PKC-ζ antibody conjugated with agarose. The samples were incubated overnight with gentle rocking, followed by centrifugation and a few washes. PKC-ζ beads without protein sample were used as negative controls. Proteins were separated by SDS-PAGE and transblotted onto a 0.2 μM nitrocellulose membrane. Immunoreactive bands were visualized using the Amersham Imager 600 software (GE Healthcare, Chalfont St Giles, UK). The association of Cx43 with PKC-ζ was confirmed by adding a Cx43 primary antibody to the blot membrane.

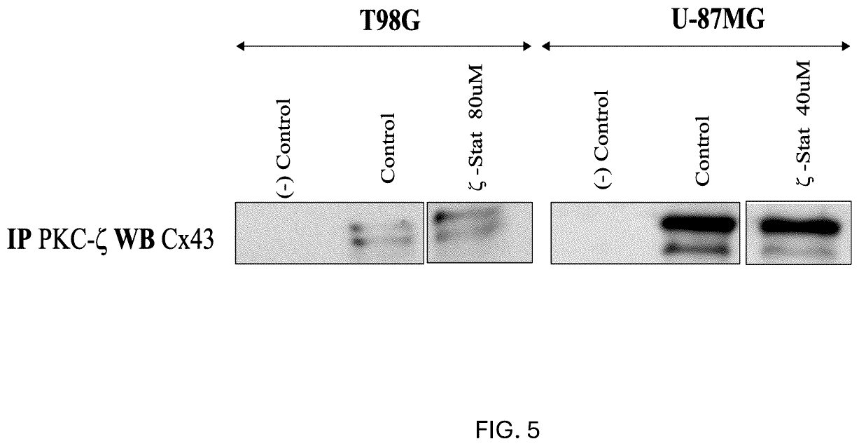

Immunoblot analysis illustrated in FIG. 5 demonstrates a strong association between PKC-ζ and Cx43 in T98G cells. In contrast, co-immunoprecipitation analysis in U-87MG cells revealed a reduced interaction between PKC-ζ and Cx43 upon inhibition of PKC-ζ mediated phosphorylation of Cx43. These findings indicate that PKC-ζ modulates Cx43 association in a cell line-dependent manner, suggesting distinct regulatory mechanisms in T98G and U-87MG cells.

### Example 5: Flow Cytometry to Assess Apoptosis

Annexin-V/APC (Annexin V/Allophycocyanin) and DAPI (4′,6-diamidino-2-phenylindole) based flow cytometry was used to assess apoptosis in the glioblastoma cells. Approximately 5×10⁴ cells were seeded into 100 mm culture flasks and treated with ζ-Stat for five days. Post treatment, cells were detached using TrypLE Express Enzyme (lx), centrifuged, and washed with PBS (phosphate-buffered saline). Subsequently, the treated and untreated cells were resuspended in 100 μL of Annexin-V/APC and DAPI solution and incubated in the dark for 10 min at room temperature. Samples analyzed using a BD FACSCanto II flow cytometer (Becton, Dickinson and Company). APC fluorescence was detected with 633 nm excitation and 700 nm emission, while DAPI was measured at 350 nm excitation and 450 nm emission. Data were analyzed using FACSDiva software v6.3.1 (BD Biosciences).

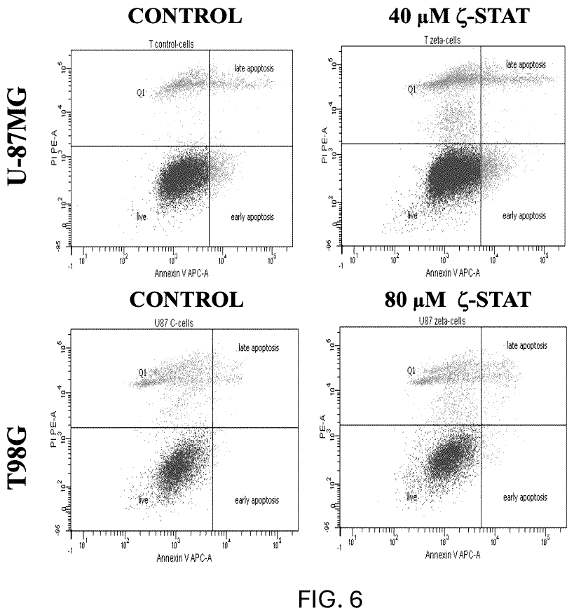

Annexin V/APC and DAPI-based flow cytometry analysis employed to evaluate apoptosis is represented in the FIG. 6. In T98G cell line, the control group exhibited 81.7% viable cells, with 5.8% in early apoptosis and 3.7% in late apoptosis. Treatment with ζ-Stat (80 μM) induced 5.9% early apoptosis and 3.6% of late apoptosis. In U-87MG cells, the control group demonstrated 71.6% viability, with 1.3% of cells in early apoptosis and 3.6% of cells in late apoptosis, while treatment with ζ-Stat (40 μM) induced 5.2% of the cells to late apoptosis and 1.7% to early apoptosis. These findings suggest that ζ-Stat treatment modestly increases early and late apoptotic cell populations in both cell lines. However, the low levels of apoptosis observe indicate that ζ-Stat may not exert its primarily effect through apoptotic pathways. Therefore, the present invention further investigates the impact of ζ-Stat on cell cycle progression.

### Example 6: β-Galactosidase Staining of Glioblastoma Cells for Senescence

Approximately 2×10³ cells were seeded in 6-well plates, followed by treatment with ζ-Stat for five days. Subsequently, cells were fixed and stained according to the manufacturer's protocol using the senescence β-Gal staining kit. Briefly, T98G and U-87MG cells were fixed with 1×β-Gal fixative for 20 min at room temperature and stained with complete 1×β-Gal stain solution in a dry incubator overnight at 37° C. Images were captured using Jenoptik Gryphax® software (Jenoptik AG) with differential interference contrast brightfield microscopy (Motic AE31E; magnification, ×20).

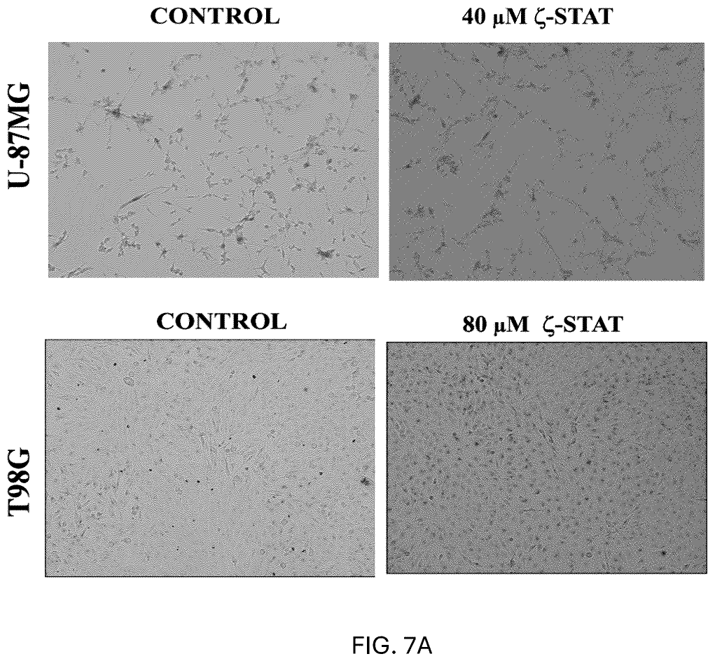

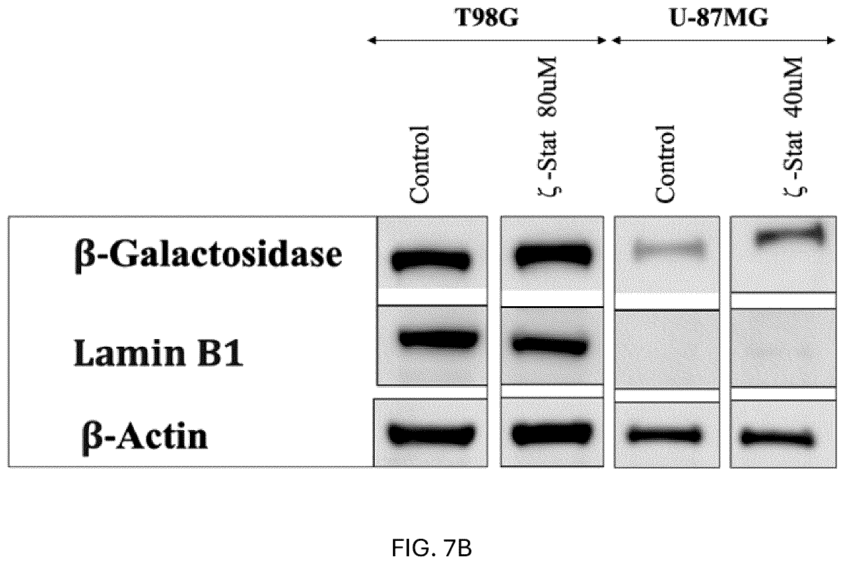

Cellular senescence was assessed by analyzing the upregulation of senescence-associated β-Galactosidase (β-Gal) and downregulation of nuclear membrane proteins [30]. Immunofluorescence analysis shown in FIG. 7A demonstrated that T98G and U-87MG cells treated with ζ-Stat for 5 days exhibited an increase in β-Gal activity. FIG. 7B illustrates that treatment with ζ-Stat resulted in a 30% increase in β-galactosidase expression in T98G cells and a 50% increase in U-87MG cells. Additionally, ζ-Stat treatment at 80 μM led to an 8% reduction in Lamin B1 expression in T98G cells, indicating disruption of nuclear membrane integrity associated with senescence. 3-actin was used as a loading control.

### Example 7: Effect of ζ-Stat on Senescence Markers

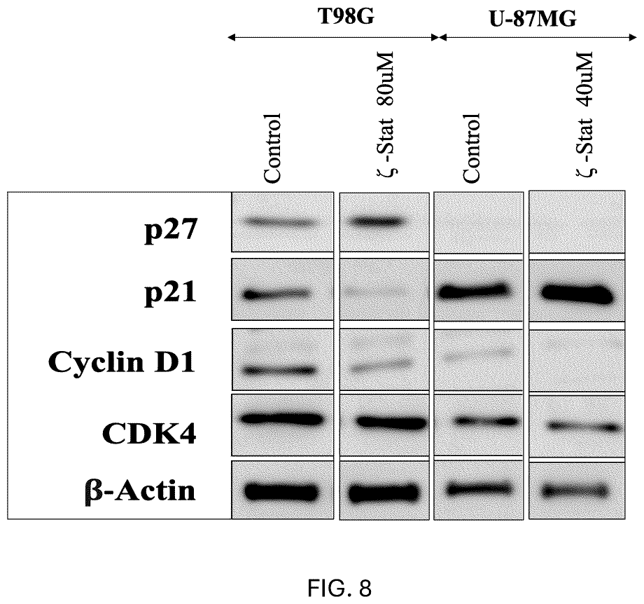

Western blots analysis was performed to evaluate the effect of ζ-Stat on senescence associated proteins involved in glioblastoma cells cycle progression. The results are illustrated in FIG. 8. The G₁ to S phase transition of cell cycle is primarily regulated by the interaction between cyclin D1 and CDK4. Cyclin-dependent kinase inhibitors such as p27 and p21 negatively regulate this process by preventing the formation of the cyclin D1-CDK4 complex, thereby inducing cell cycle arrest [31]. In T98G cells treated with ζ-Stat (80 M), p27 expression increased by 32%, while CDK4 and cyclin D1 levels were reduced by 6% and 53%, respectively. In contrast, U-87 MG cells treated with ζ-Stat (40 M) exhibited a reduction in p21 expression by 18%. Nevertheless, CDK4 and cyclin D1 protein levels still decreased by 48% and 64%, respectively. β-actin was used as a loading control.

### Example 8: Effect of Inhibitors in PI3K/AKT Signaling

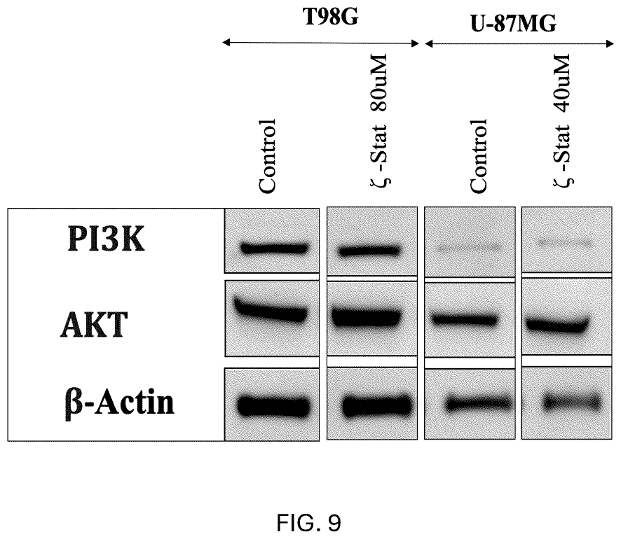

The PI3K/AKT pathway is a critical driver of glioblastoma cell growth, survival, and invasive behavior. To determine the effects of ζ-Stat on this signaling, protein expression levels were analyzed by western blot (FIG. 9). In T98G cells, treatment with ζ-Stat (80 μM) cell line led to a 27% decline in PI3K, whereas AKT levels showed a modest decrease of 6%. In U-87MG, ζ-Stat (40 M) lowered PI3K expression by 17% and AKT by 33%. These findings indicate that ζ-Stat may inhibit PI3K/AKT pathway. β-actin served as a loading control in these experiments.

### Example 9: Effect of Inhibitors in EMT Markers

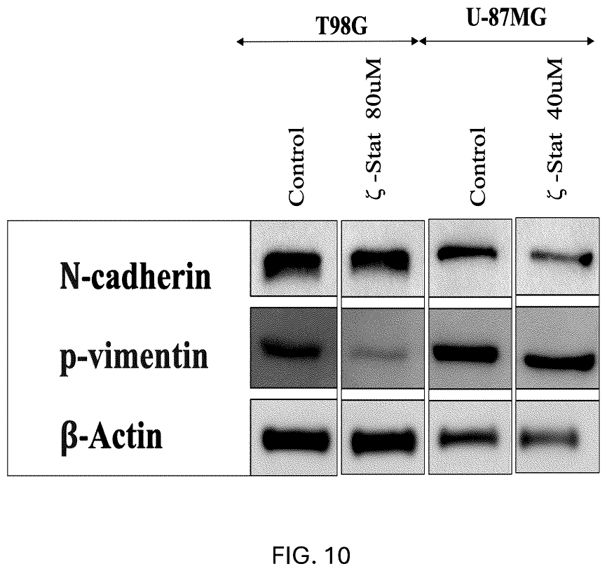

The effects of PI3K/AKT pathway inhibition on EMT markers are shown in FIG. 10. Treatment with ζ-Stat caused a slight 4% increase in N-cadherin expression in T98G cells, whereas in U-87MG cells, N-cadherin levels were markedly reduced by 46%. Furthermore, phosphorylated vimentin levels decreased by 21% in T98G cells and 26% in U-87MG cells after ζ-Stat administration. These findings indicate that suppression of PI3K/AKT signaling and Connexin 43 activity disrupts EMT-related processes. The notable decreases in N-cadherin and phospho-vimentin expression highlight ζ-Stat potential to reduce the migratory and invasive characteristics of glioblastoma cells.

### REFERENCES

[1] Perry, A.; Wesseling, P. Histologic classification of gliomas. Handb. Clin. Neurol. 2016, 134, 71-95.
[2] Batash, R.; Asna, N.; Schaffer, P.; Francis, N.; Schaffer, M. Glioblastoma Multiforme, Diagnosis and Treatment; Recent Literature Review. Curr. Med. Chem. 2017.
[3] Tran, B.; Rosenthal, M. A. Survival comparison between glioblastoma multiforme and other incurable cancers. J. Clin. Neurosci. 2010, 17, 417-421.
[4] Wu, W., Klockow, J. L., Zhang, M., Lafortune, F., Chang, E., Jin, L., Wu, Y., & Daldrup-Link, H. E. Glioblastoma multiforme (GBM): An overview of current therapies and mechanisms of resistance, Pharmacological research. 2021, 171, 105780,
[5]H. J. Mackay, C. J. Twelves. Targeting the protein kinase C family: are we there yet?Nat Rev Cancer. 2007, pp. 554-562
[6] Baldwin, R., Parolin, D. & Lorimer, L. Regulation of glioblastoma cell invasion by PKCι and RhoB. Oncogene. 2008, 27, 3587-3595.
[7] Donson, A. M., Banerjee, A., Gamboni-Robertson, F. et al. Protein Kinase C Isoform is Critical for Proliferation in Human Glioblastoma Cell Lines, J Neurooncol. 2000, 47, 109-115.
[8] Guo H, Gu F, Li W, Zhang B, Niu R, Fu L, Zhang N, Ma Y. Reduction of protein kinase C zeta inhibits migration and invasion of human glioblastoma cells. J Neurochem. 2009.
[9] Kumar N M, Gilula N B. The gap junction communication channel. Cell, 1996.
[10]J. H. Lin, T. Takano, M. L. Cotrina, G. Arcuino, J. Kang, S. Liu, Q. Gao, L. Jiang, F. Li, H. Li chtenberg-Frate, S. Haubrich, K. Willecke, S. A. Goldman, M. Nedergaard. Connexin 43 enhances the adhesivity and mediates the invasion of malignant glioma cells. J. Neurosci., 22. 2002, pp. 4302-4311.
[11]W. Zhang, C. Nwagwu, D. M. Le, V. W. Yong, H. Song, W. T. Couldwell. Increased invasive capacity of connexin43-overexpressing malignant glioma cells. J. Neurosurg., 99. 2003, pp. 1039-1046.
[12] Gielen, P. R.; Aftab, Q.; Ma, N.; Chen, V. C.; Hong, X.; Lozinsky, S.; Naus, C. C.; Sin, W. C. Connexin43 Confers Temozolomide Resistance in Human Glioma Cells by Modulating the Mitochondrial Apoptosis Pathway. Neuropharmacology 0.2013, 75, 539-548.
[13] Theis, M.; Giaume, C. Connexin-Based Intercellular Communication and Astrocyte Heterogeneity. Brain Res. 2012, 1487, 88-98.
[14] Pridham, K. J., Shah, F., Hutchings, K. R. et al. Connexin 43 confers chemoresistance through activating PI3K. Oncogenesis. 2022, 11, 2.
[15] Lin, J. H., Takano, T., Cotrina, M. L., Arcuino, G., Kang, J., Liu, S., Gao, Q., Jiang, L., Li, F., Lichtenberg-Frate, H., Haubrich, S., Willecke., K., Goldman, S. A., & Nedergaard, M. Connexin 43 enhances the adhesivity and mediates the invasion of malignant glioma cells. The Journal of neuroscience: the official journal of the Society for Neuroscience. 2002, 22(11), 4302-4311.
[16] Axelsen L N, Calloe K, Holstein-Rathlou N H, Nielsen M S. Managing the complexity of communication: regulation of gap junctions by post-translational modification. Front Pharmacol. 2013, 4:130
[17] Bao X, Altenberg G A, Reuss L. Mechanism of regulation of the gap junction protein connexin 43 by protein kinase C-mediated phosphorylation. Am J Physiol Cell Physiol. 2004; 286(3):C647-654.
[18] Zou J, Yue X Y, Zheng S C, Zhang G, Chang H, Liao Y C, Zhang Y, Xue M Q, Qi Z. Cholesterol modulates function of connexin 43 gap junction channel via PKC pathway in H9c2 cells. Biochim Biophys Acta. 2014; 1838(8):2019-25.
[19] Solan J L, Fry M D, TenBroek E M, Lampe P D. Connexin43 phosphorylation at S368 is acute during S and G2/M and in response to protein kinase C activation. J Cell Sci. 2003; 116:2203-11
[20] Nimlamool, W.; Andrews, R. M. K.; Falk, M. M. Connexin43 Phosphorylation by PKC and MAPK Signals VEGF-Mediated Gap Junction Internalization. Mol. Biol. Cell. 2015, 26 (15), 2755-2768.
[21] Gould V E, Mosquera J M, Leykauf K, Gattuso P, Dürst M, Alonso A. The phosphorylated form of connexin43 is up-regulated in breast hyperplasias and carcinomas and in their neoformed capillaries. Hum Pathol. 2005; 36:536-45.
[22] Solan J L, Hingorani S R, Lampe P D. Cx43 phosphorylation sites regulate pancreatic cancer metastasis. Oncogene. 2021; 40:1909-20.
[23] Ye X Y, Jiang Q H, Hong T, Zhang Z Y, Yang R J, Huang J Q, Hu K, Peng Y P. Altered expression of connexin43 and phosphorylation connexin43 in glioma tumors. Int J Clin Exp Pathol. 2015; 8:4296-306.
[24] Cheng, C. K., Fan, Q. W., & Weiss, W. A. PI3K signaling in glioma-animal models and therapeutic challenges. Brain pathology (Zurich, Switzerland). 2009, 19(1), 112-120.
[25] Guo, H., Gu, F., Li, W., Zhang, B., Niu, R., Fu, L., Zhang, N., & Ma, Y. Reduction of protein kinase C zeta inhibits migration and invasion of human glioblastoma cells. Journal of neurochemistry. 2009, 109(1), 203-213.
[26] Harder, B. G., Peng, S., Sereduk, C. P. et al. Inhibition of phosphatidylinositol 3-kinase by PX-866 suppresses temozolomide-induced autophagy and promotes apoptosis in glioblastoma cells. Mol Med. 2019, 25, 49.
[27] Maharati, A., Moghbeli, M. PI3K/AKT signaling pathway as a critical regulator of epithelial-mesenchymal transition in colorectal tumor cells. Cell Commun Signal. 2023, 21, 201.
[28] Kotini, M., Barriga, E. H., Leslie, J., Gentzel, M., Rauschenberger, V., Schambony, A., & Mayor, R. Gap junction protein Connexin-43 is a direct transcriptional regulator of N-cadherin in vivo. Nature communications. 2018, 9(1), 3846.
[29] Kotini, M., Barriga, E. H., Leslie, J. et al. Gap junction protein Connexin-43 is a direct transcriptional regulator of N-cadherin in vivo. Nat Commun. 2018, 9, 3846.
[30] Wang A S, Ong P F, Chojn owski A, Clavel C, Dreesen O. Loss of lamin 131 is a biomarker to quantify cellular senescence in photoaged skin. Sci Rep. 2017; 7:156⁷8.
[31]. Sherr C J, Roberts J M. Living with or without cyclins and cyclin-dependent kinases. Genes Dev. 2004; 18:2699-2711.

We claim:

1. A method of treating glioma or glioblastoma comprising administering to a subject in need an effective amount of a phosphokinase C ζ (PKC-ζ) inhibitor, wherein the PKC-ζ inhibitor is 8-hydroxy-1,3,6-naphthalenetrisulfonic acid (ζ Stat).

2. The method of claim 1, wherein the ζ Stat has the formula of Formula I:

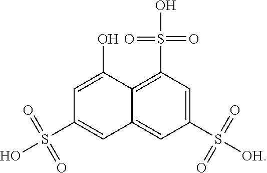 [SMILES: O=S(=O)(O)c1cc(O)c2c(S(=O)(=O)O)cc(S(=O)(=O)O)cc2c1]

3. The method of claim 1, wherein the subject is a human.

4. The method of claim 1, wherein the subject has a brain glioma.

5. The method of claim 1, wherein the subject has glioblastoma multiforme.

6. The method of claim 1, wherein the ζ Stat is in a composition or a pharmaceutical composition.

7. The method of claim 1, wherein the pharmaceutical composition further comprises one or more pharmaceutically acceptable carrier.

8. A method of reducing or inhibiting the proliferation of glioma or glioblastoma cells, comprising delivering to a glioma cell or glioblastoma cell an effective amount of a PKC-ζ inhibitor, wherein the PKC-ζ inhibitor is 8-hydroxy-1,3,6-naphthalenetrisulfonic acid (ζ Stat).

9. The method of claim 8, wherein the ζ Stat has the formula of Formula I:

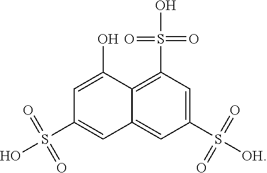 [SMILES: O=S(=O)(O)c1cc(O)c2c(S(=O)(=O)O)cc(S(=O)(=O)O)cc2c1]

10. The method of claim 8, wherein the ζ Stat is delivered to a subject with glioblastoma.

11. The method of claim 8, wherein the subject is a human.

12. The method of claim 8, wherein the method induces senescence of the glioma or glioblastoma cells.

13. A method of inhibiting the expression of protein kinase C-ζ (PKC-ζ) in a glioma or glioblastoma cell comprising delivering to a cell an effective amount of a PKC-ζ inhibitor, wherein the PKC-ζ inhibitor is 8-hydroxy-1,3,6-naphthalenetrisulfonic acid (ζ Stat).

14. The method of claim 13, wherein the cell is a human cell.

15. The method of claim 13, wherein the ζ Stat inhibits Phosphoinositide 3-Kinase/Protein Kinase B (PI3K/AKT) pathway in the cell.

16. The method of claim 13, wherein the ζ Stat reduces activation of Connexin 43 in the cell.

17. The method of claim 13, wherein the method induces senescence in the glioma or glioblastoma cells.

18. The method of claim 13, wherein the method inhibits epithelial-mesenchymal transition in the glioma or glioblastoma cell.

19. The method of claim 13, wherein effective amount of ζ Stat is about 40 uM to about 80 uM.
